(19) United States
(12) Patent Application Publication
(10) Pub. No.: US 2006/0052421 A1
(43) Pub. Date: Mar. 9, 2006

(54) Conjugation agent

(75) Inventors: Thomas R. Welter, Webster, NY (US); Kevin P. Dockery, Rochester, NY (US)

Correspondence Address: Paul A. Leipold;Patent Legal Staff, Eastman Kodak Company, 343 State Street, Rochester, NY, 14650-2201, US

(73) Assignee: Eastman Kodak Company

(21) Appl. No.: 10/936,908

(22) Filed: Sep. 9, 2004

(51) Int. Cl. C07K 14/47; C07D 213/63; C07D 403/02; A61K 31/4439; A61K 31/4412

(52) U.S. Cl. 514/341; 514/345; 546/268.4; 530/409; 546/290

(57) ABSTRACT

Conjugation agents of the formula: 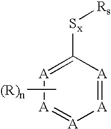
are described, wherein Sₓ is —S—, —SO— or —SO₂—; Rₛ is a carbon-containing substituent that does not include a cyano group; each R is a substituent, with at least one R having a reaction site that is not a carboxylic acid; n is 0-5; each A is carbon or nitrogen, with the proviso that no more than three A can be nitrogen; and Sₓ—Rₛ is a leaving group. The conjugation agents have good thiol reactivity and selectivity, and good stability with regard to hydrolysis.

### FIELD OF THE INVENTION

The present invention relates to aromatic conjugation agents and their use, for example, in conjugation of biomolecules through aromatic cross-linking.

### BACKGROUND OF THE INVENTION

Conjugation refers to the covalent chemical attachment of two or more targets, wherein each target independently can be biological or chemical. Conjugation is also known as linking, cross-linking, or ligation. When conjugation involves a compound from a biological source, or affords a material of biological usefulness, it can be termed “bioconjugation.” The conjugation agent bound to one or more target is referred to as a “conjugate.”

The science and art of bioconjugation is well established and documented, for example, in Chemistry of protein Conjugation and Cross-linking by S. S. Wong, CRC Press, Boston (1991), and Bioconjugate Techniques by G. T. Hermanson, Academic Press, San Diego (1996). Bioconjugation involves the coupling of a target of biological significance, for example, a protein, a polynucleotide, a hormone, an antigen, an enzyme, a co-factor, or other molecule, with another biological molecule or site, or with a chemical, such as a drug, a dye, a radioactive tag, a fluorescent tag, an activating agent, a support, another conjugating group, or other chemical group. The products of bioconjugations are of use in disparate fields including, but not limited to, chemotherapeutics, clinical diagnostics, molecular biological research, catalyst formulations, materials research, pharmacology, and the like. Bioconjugation can occur through reactive groups, such as amine- and thiol-containing groups.

Many conjugation agents are known, as described, for example, by G. T. Hermanson in Bioconjugate Techniques. However, only a limited number of conjugation agents are known to be reactive toward thiols. These conjugation agents can include alkylating agents; activated olefins such as maleimides and acrylic acid derivatives; disulfides; and arylation agents. Each of these classes of conjugation agents lacks complete selectivity, is too unreactive to be of wide practical use, or both.

For example, arylation reactions based upon nucleophilic aromatic substitution (S_{N}Ar) chemistry are not selective enough to function efficiently in conjugation agents. Such arylation reactions include an electron poor, carbocyclic, haloaromatic compound (1, X=halogen atom, EWG=electron withdrawing group) reacting with a nucleophilic site on a target to provide an arylated product (2, Z=a heteroatom, e.g. N, S, or O).

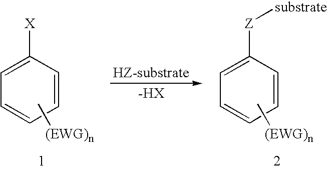

These compounds display little selectivity. They can bind nitrogen, sulfur, and even oxygen groups (S. S. Wong, Chemistry of protein Conjugation and Cross-linking). Such molecules have been used as nitrogen tagging agents. The order of reactivity of such aromatic substrates follows the trend X═F>Cl˜Br>I>SO₃⁻, and the rate of substitution increases with increasing electron withdrawal from the aromatic ring. Examples of this chemistry can be found in S. Shaltiel, “Dinitrophenylation and Thiolysis as a Tool in Protein Chemistry,” Isr. J. Chem., 12(1-2), 403-19 (1974); S. Shaltiel and M. Tauber-Finkelstein, “Introduction of an Intramolecular Crosslink at the Active Site of Glyceraldehyde 3-Phosphate Dehydrogenase,” Biophys. Res. Commun., 44(2), 484-90 (1971); S. Shaltiel and M. Soria, “Dinitrophenylation and Thiolysis in the Reversible Labeling of a Cysteine Residue Associated with the Nicotinamide Adenine Dinucleotide Site of Rabbit Muscle Glyceraldehyde-3-phosphate Dehydrogenase,” Biochemistry, 8(11), 4411-I5 (1969); and S. Shaltiel, “Thiolysis of some Dinitrophenyl Derivatives of Amino Acids,” Biochem. Biophys. Res. Commun, 29(2), I78-83 (1967).)

Some conjugation agents known in the art exhibit satisfactory chemoselectivity for thiols, but are limited in their usefulness by their poor stability towards acid or base catalyzed hydrolysis reactions, polymerizations, or nucleophilic ring openings. Examples of such known conjugation agents include N-substituted maleimides, which show high selectivity for thiols over amines (kᵣₑₗ(thiol)/kᵣₑₗ(amine)>100,000), but are subject to hydrolysis (lifetime at room temperature and neutral pH values ˜1 day). For related studies of N-alkylmaleimides, see S. Hashida et al., Appl. Biochem., 6, 56-63 (1984); P. Knight, Biochem. J., I79, 191-197 (1979); M. N. Khan, J. Chem. Soc., Perkin 2, 819-828 (1987); M. N. Khan, J. Chem. Soc., Perkin 2, 1977-1985 (1985); M. N. Khan, J. Chem. Soc., Perkin 2, 891-897 (1985); and S. Matsui and H. Aida, J. Chem. Soc., Perkin 2, 1277-1280 (1978).

Mixed aromatic sulfides having one reactive site are described in the art, for example, in Welter, J. Soc. Photogr. Sci. Technol. Japan, Vol. 62, No. 2, 98-105 (1999); Münch et al., Bioorganic & Medicinal Chemistry, 11, 2041-2049 (2003); U.S. Pat. No. 5,478,711; U.S. Pat. No. 5,567,577; and U.S. Pat. No. 5,460,932. These would not be useful for use as conjugating agents without further manipulation.

It is desirable to provide conjugation agents that are highly selective for and reactive with thiol-containing groups. It is further desirable to provide a conjugation agent that is stable in aqueous solution. More selective and efficient methods for thiol-conjugation that can be used in aqueous solutions are needed.

### SUMMARY OF THE INVENTION

Conjugation agents having the formula: 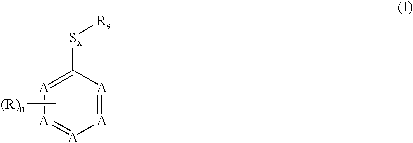
are disclosed, and their uses, wherein Sₓ is —S—, —SO— or —SO₂—; Rₛ is a carbon-containing substituent that does not include a cyano group; each R is a substituent, with at least one R having a reaction site that is not a carboxylic acid; n is 0-5; and each A is carbon or nitrogen, with the proviso that no more than three A can be nitrogen.

### Advantages

Conjugation agents having improved reactivity with and selection for thiol-containing groups are disclosed, and uses thereof, such as in bioconjugation. Such conjugation agents can have higher rates of reactivity and improved selectivity, and can be stable in aqueous solutions.

### DETAILED DESCRIPTION OF THE INVENTION

For conjugation, a conjugation agent, also called a linking group or ligand, is used. The conjugation agent can be an organic molecule with two or more reactive sites, wherein each reactive site can covalently bond to at least one target. “Target” as used herein includes chemical, biochemical, and biological structures, for example, dyes, radioactive tags, fluorescent tags, activating agents, supports, other conjugating groups, proteins, polynucleotides, hormones, antigens, enzymes, co-factors, other chemical or biological structures, or biological activation sites. The resultant conjugate can include two or more targets of interest, each of which is covalently attached to the conjugation agent through a reactive group on the conjugation agent. The reactive groups of the conjugation agent can bond to heteroatom functional groups on the target. Such target functional groups can include, for example, amines, carboxylic acids, phenols, alcohols, acidic nitrogen heterocycles, thiolates, and thiols, also referred to as mercaptans or sulfhydryls. At least one target includes a thiol or thiolate group.

Target functional groups that do not have sufficiently reactive groups can be prepared for conjugation via an activation process. For example, antibodies are held together via disulfide bonds which, when reduced, can provide more reactive thiol groups. Targets can be derivatized prior to conjugation to add or alter the reactivity of pendant functional groups, as described, for example, in Chemistry of protein Conjugation and Cross-linking by S. S. Wong. Any technique of activation or derivatization known in the art can be used to prepare a target for conjugation.

If the targets to be joined through the conjugation agent bear the same or similar reactive groups, a conjugation agent bearing two or more of the same reactive sites can be employed. Such conjugation agents are called homofunctional conjugation agents. If the reactive groups of compounds to be connected are different, the conjugation agent can bear two or more different reactive groups. This is called a heterofunctional conjugation agent.

Targets can bear multiple reactive sites, with varying degrees of reactivity for each site based on the properties of the reactive group at the site, and steric hindrance. For effective and reproducible conjugation, a single site, or two or more closely spaced sites on a single target, can be chosen for conjugation. When two or more closely spaced sites are chosen for reaction with the conjugation agent, the conjugation agent can be referred to as a bidentate or multi-binding ligand.

The conjugation agent can be chosen to selectively react with one or more functional group on the target. The conjugation agent can be chemoselective. Chemoselectivity of a conjugation agent for a specific target functional group can be determined by examination of differences in rate constants for covalent bonding of the conjugation agent to the target functional group. The rate constant for reaction of the conjugation agent with a desired target functional group should be greater than that for reaction with an alternate target functional group.

The ratio of the rate constants of respective functional groups is a measure of selectivity. Selectivity of a reaction can be affected by reducing the rate constant of the reaction, for example, by lowering the reaction temperature, by use of a poor solvent, by use of a weaker agent, or other methods known in the art. While these sorts of changes can lead to perceptible improvements in selectivity, they can also slow reaction rates, which can lead to prohibitively long reaction times or lower overall yield of conjugates. There is a need to balance selectivity of a reaction with the resultant reaction rate.

Conjugates can be formed at one time, or in a series of reactions, wherein first one reactive site, then another, is bound to a desired target, or to an additional conjugation agent. Selective chemistry allows for multi-part reactions to form a conjugate.

Functional groups that can covalently bond two desired targets, directly or through a linking agent, can include amines, thiolates, and thiols, for example. Amine groups are present in most biological targets, and can be present in chemical compounds and inorganic substrates used with biological targets. Thiol-containing groups, while less prevalent, are found in many biological targets, can be added to functionalize various tags, and can be present in chemical targets. It can be advantageous to form a conjugate in a multi-part reaction by binding first one target, than a second target. It can be advantageous for the targets to be bound through different chemistries, such as by using a heterofunctional linker or conjugating agent. For example, one target can be linked through an amine-containing group, and one target can be linked through a thiol-containing group. For example, gelatin coated on a solid surface, such as glass or plastic, can be used as a substrate for binding proteins or peptides. The gelatin can have lysine groups pendant, and the lysine groups can be functionalized by application of an aqueous solution of a heterofunctional conjugating agent, binding the conjugating agent to the target gelatin. Suitable heterofunctional binding agents are known in the art. The conjugating agent can then react with a second target through a second linking group, such as a thiol-containing group. For such a reaction scheme to be effective, the thiol-specific portion of the heterofunctional agent must (1) not react with the gelatin amine sites, (2) survive the aqueous reaction conditions of that amine ligation, and (3) react selectively with the thiol-containing group of the second target, for example, a glutathione. Selectivity, stability, and reactivity are necessary at one or more reaction sites to enable the conjugation agent to effectively covalently bind two or more targets.

Suitable conjugation agents can have the following formula I: 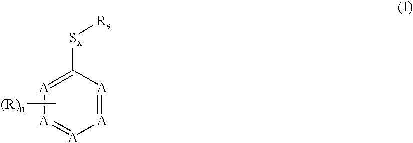
wherein Sₓ is —S—, —SO— or —SO₂—; Rₛ is a carbon-containing group that does not include a cyano group; each R is a substituent, with at least one R having a reaction site that is not a carboxylic acid; n is 0-5; each A is carbon or nitrogen, with the proviso that no more than three A can be nitrogen; and Sₓ—Rₛ is a leaving group.

According to certain embodiments, the structure of the conjugation agent can be as shown in formula II: 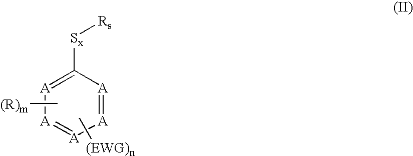
wherein Sₓ is —S—, —SO— or —SO₂—; Rₛ is a carbon-containing group that does not include a cyano group; each R is a substituent, with at least one R having a reaction site that is not a carboxylic acid; each EWG is a substituent that includes an electron withdrawing group; n is 0-5; m is 0-4; m+n is less than or equal to 5; each A is carbon or nitrogen, with the proviso that no more than three A can be nitrogen, and the proviso that when all A are carbon, n is greater than or equal to 1; and Sₓ—Rₛ is a leaving group. The above formulas I and II represent conjugation agents with high selectivity and good reaction rates.

In the above formulas I and II, the site of attachment for leaving group SₓRₛ is referred to herein as the “primary reaction site.” This site can react with a sulfur-containing functional group on a target. The sulfur-containing functional group can be a thiol or thiolate.

Sₓ can be a sulfur molecule in any oxidation state capable of linkage to at least two substituents. For example, Sₓ can be a sulfide, a sulfone, or a sulfoxide. According to certain embodiments, Sₓ can be sulfide.

Rₛ can be a carbon-containing substituent group, with the proviso that Rₛ is not cyano. Rₛ can include at least one nitrogen. Rₛ can be a substituted or unsubstituted, straight or branched aromatic; or a substituted or unsubstituted, straight or branched, non-aromatic, heterocyclic or non-hetereocyclic substituent. According to certain embodiments, Rₛ can be a substituted or unsubstituted heterocyclic aromatic substituent. Rₛ can be a single or multiple aromatic or heterocyclic ring structure, wherein the multiple ring structures are five rings or less, and any two-ring structures are not fused. Rₛ can be a substituted or unsubstituted nitrogen-containing heterocyclic substituent, for example, pyridine, pyrimidine, triazine, quinoline, isoquinoline, pyrrol, imidazole, pyrazole, oxazole, isoxazole, thiazole, isothiazole, triazoles, thiadiazoles, oxadiazoles, or tetrazole. According to certain embodiments, Rₛ can be a tetrazolyl group, an N-alkyltetrazolyl group, or an N-ethyltetrazolyl group. Rₛ can react with a first heteroatom-containing target.

Ring member A can be carbon or nitrogen, so long as not more than three A are nitrogen. When one or more A is a substituted or unsubstituted nitrogen, A is considered to be an electron withdrawing group, and any substituents R need not be electron withdrawing. When one or more A is a nitrogen, a substituent R can be associated with the nitrogen A. According to certain embodiments, a substituent R from nitrogen A does not include an electron withdrawing group, because the nitrogen A can function as an electron withdrawing group. For example, a substituent R of nitrogen A can include a branched or unbranched, substituted or unsubstituted alkyl, or R can be a substituted or unsubstituted aromatic or heteroaromatic. Examples of suitable aza-analogues formed when one or more A is nitrogen can include, but are not limited to, pyridine, pyridinium, pyrimidine, pyrimidinium, triazine, and triazinium.

In the above formulas I and II, each R can be any substituent group, without limitation. Suitable substituents can include, for example, solubilizing groups; fluorescent, chemoluminescent, luminescent, or radioactive tags; reactive functionalites; and electron withdrawing groups. Examples of substituent groups include, but are not necessarily limited to, hydrogen; a linear or branched, saturated or unsaturated alkyl group of 1 to 20 carbon atoms, for example, 1 to 10 carbon atoms, such as but not limited to methyl, ethyl, n-propyl, isopropyl, t-butyl, hexyl, decyl, benzyl, methoxymethyl, hydroxyethyl, iso-butyl, or n-butyl; alkenyl of 2 to 10 carbon atoms; alkynyl of 2 to 10 carbon atoms; alkylhalo; a substituted or unsubstituted aryl group of 3 to 14 carbon atoms, for example, 5-10 carbon atoms, such as but not limited to phenyl, naphthyl, anthryl, tolyl, xylyl, 3-methoxyphenyl, 4-chlorophenyl, 4-carbomethoxyphenyl or 4-cyanophenyl; a substituted or unsubstituted cycloalkyl group of 3 to 14 carbon atoms, for example, 5 to 14 carbon atoms, such as but not limited to cyclopentyl, cyclohexyl, or cyclooctyl; a substituted or unsubstituted, saturated or unsaturated, heterocyclic group of 5-I5 atoms, for example, pyridyl, pyrimidyl, morpholino, or furanyl; cycloalkenyl; two or more rings, where any two rings can be fused or non-fused; alkoxy; aldehyde; epoxy; hydrazide; vinyl sulfone; succinimidyl ester; carbodiimide; maleimide; dithio; iodoacetyl; isocyanate; isothiocyanate; aziridine; carboxy-containing group; amino-containing group; chloromethyl; cyano; phosphate; phosphonate; sulfate; sulfonate; carboxylate; fluorophore; radioactive tag; or an affinity tag. For example, tag systems can include, but are not limited to, streptavidin and biotin, histidine tags and nickel metal ions, and glutathione-S-transferase and glutathione. Where R is a ring or ring system, each ring can be a 5- or 6-membered ring.

The substituent group R can include one or more of the following chemical groups: a single bond, a carbon atom, an oxygen atom, a sulfur atom, a carbonyl group 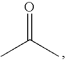
a carboxylic ester group 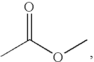
a carboxylic amide group 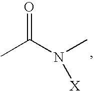
a sulfonyl group 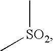
a sulfonamide group 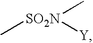
an ethyleneoxy group, a polyethyleneoxy group, or an amino group 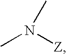
where substituents X, Y, and Z are each independently selected from the substituent groups listed above. When R includes an electron withdrawing group, it can include, but is not limited to, one or more of —NO₂, —CN, or a sulfonamide.

At least one substituent R or EWG includes a reaction site, referred to herein as the “secondary reaction site,” capable of reacting with a second heteroatom-containing target. The second heteroatom-containing target can react with a nitrogen-, sulfur-, or oxygen-containing group on the conjugating agent. The second target can be conjugated to the conjugation agent prior to, subsequent to, or concomitant with conjugation of the conjugation agent to the first, thiol-containing, target at the primary reaction site of the conjugation agent. For example, the amine group of a lysine in gelatin could be bonded to the conjugating agent via a nitrogen specific binding site contained in an R or EWG, then the first reaction site can be reacted with glutathione, completing conjugation of glutathione to gelatin through the conjugation agent.

In the above formula II, each EWG is an electron-withdrawing group. One or more EWG can contain a nitrogen. Each EWG can be independently selected from any electron-withdrawing substituent, including those listed for R. The EWG can have a positive para-substituent Hammett constant (sigma, σ). For compilations of para-substituent Hammett constants, et al., see C. Hansch, A. Leo, and D. Hoekman, Exploring QSAR: Vol. 2, Hydrophobic, Electronic, and Steric Constants; American Chemical Society: Washington D.C., 1995. Suitable electron withdrawing groups can include, but are not limited to, nitro, nitroso, cyano, sulfonyl, sulfoxyl, carbonyl, carboxaldehydo, carboalkoxy, carboaryloxy, carbonamido, sulfonamido, fluoroalkyl, fluoroaryl, azo, and azoxy groups.

R or EWG, independently, can, with two As, form a ring, or a multi-ring structure. Where R or EWG is a ring or multi-ring structure, with or without As, each ring can be a 5-membered or 6-membered ring. Smaller or larger rings can also be used.

The conjugation agent as described herein functions as a linker between two or more targets, wherein each target independently can be a biological or chemical compound. More than one conjugation agent can function as a linker, as shown in formula III:
X—(L)ₚ—Y (III)
wherein each L is a conjugation agent as defined in formula I or II, and each L can be the same or different from at least one other L; X and Y are each independently selected compounds to be joined by L; and p is 1-4.

When the conjugation agent, acting as a linker, has joined two compounds together, the resulting conjugation can have the following formula IV: 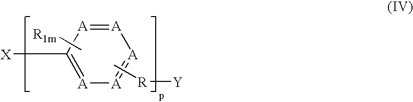
wherein R is a substituent having a reaction site as defined with regard to formulas I and II; each R₁ is a substituent, and each R₁ independently can have a reaction site, can be electron withdrawing, or can be any substituent as defined for R above with regard to formulas I and II; m is 0-4; each A is carbon or nitrogen, with the proviso that no more than three A can be nitrogen; X and Y are each are independently selected compounds bound to L; and p is 1-4. Note that the leaving group Sx-Rs is gone, having been replaced by target X. According to certain embodiments, more than one substituent group can have a reaction site, and can be bound to a compound of interest. For example, in formula IV above, one or more of the R₁ can be replaced by R—Y, wherein each R can be the same or different, and each Y can be the same or different.

Exemplary conjugation agents according to formulas I or II can include, but are not limited to, the following:

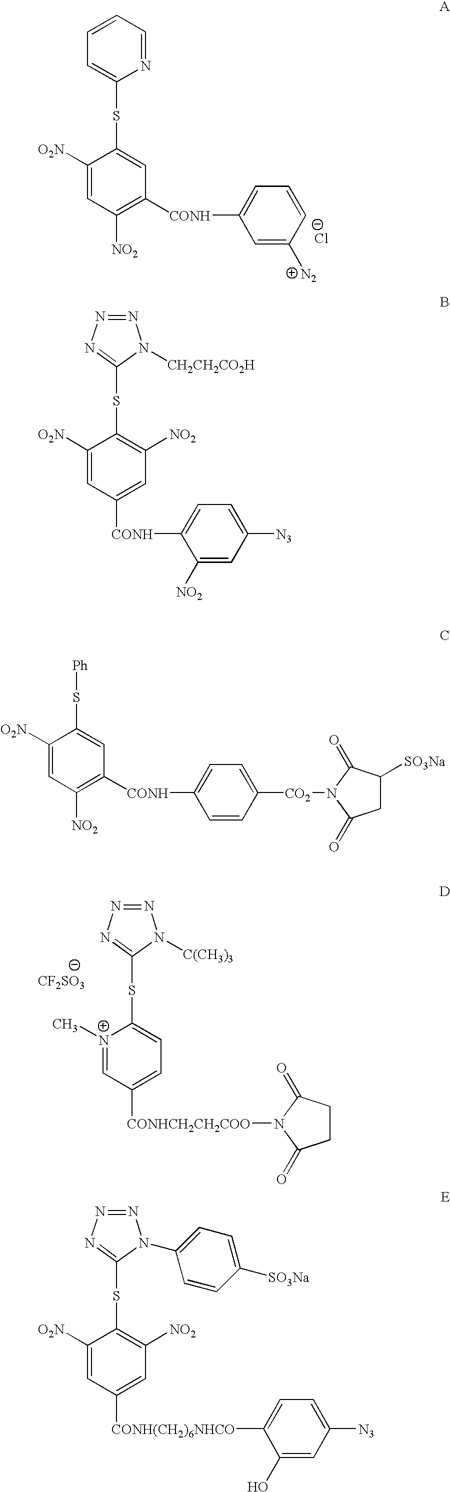

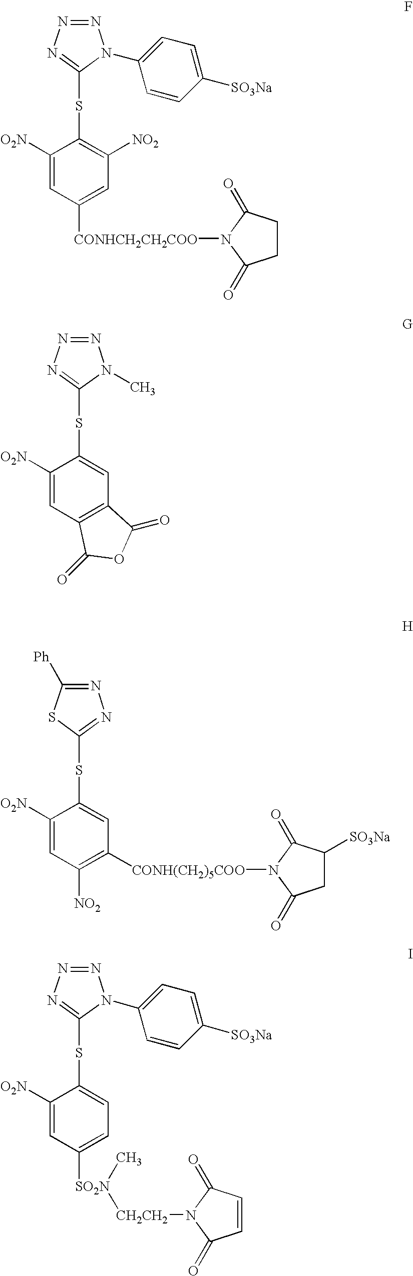

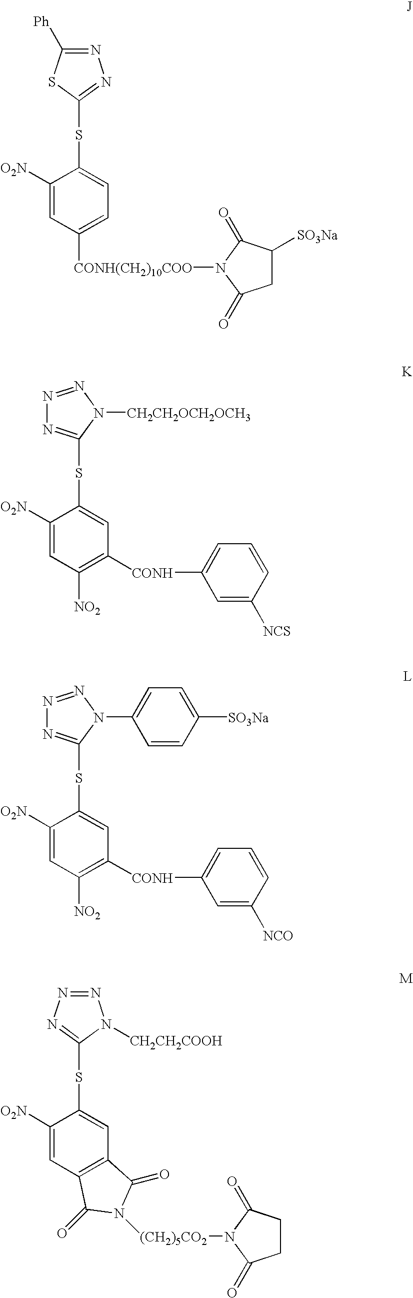

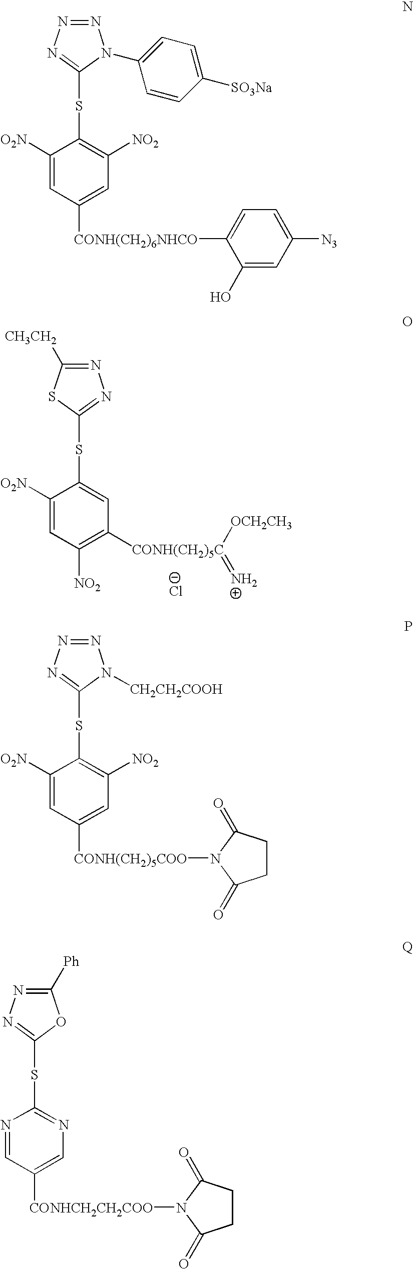

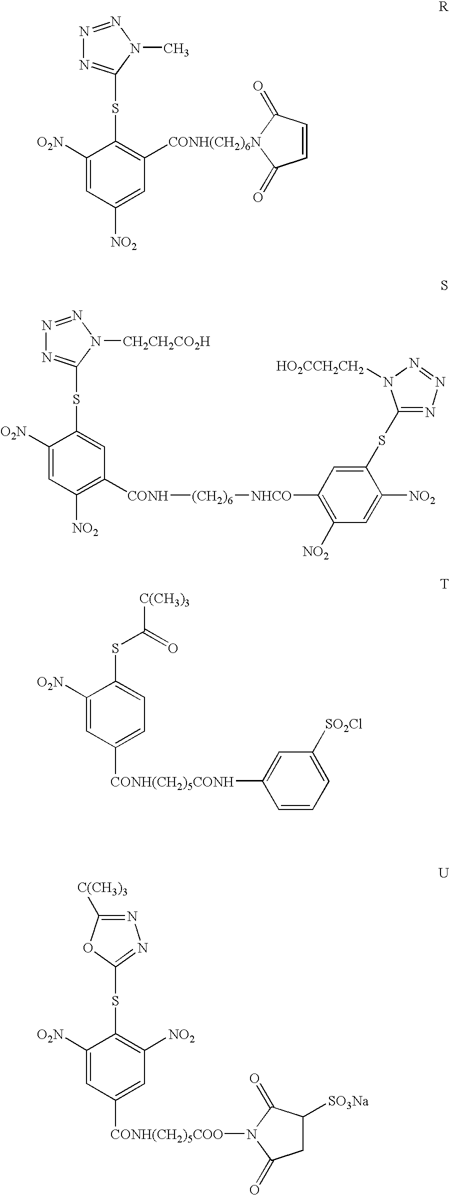

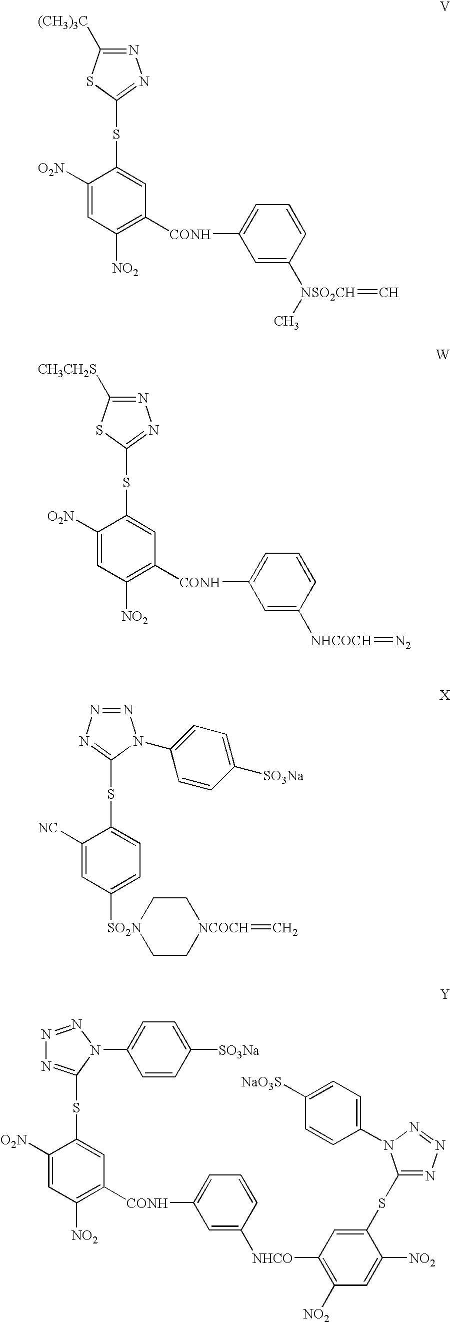

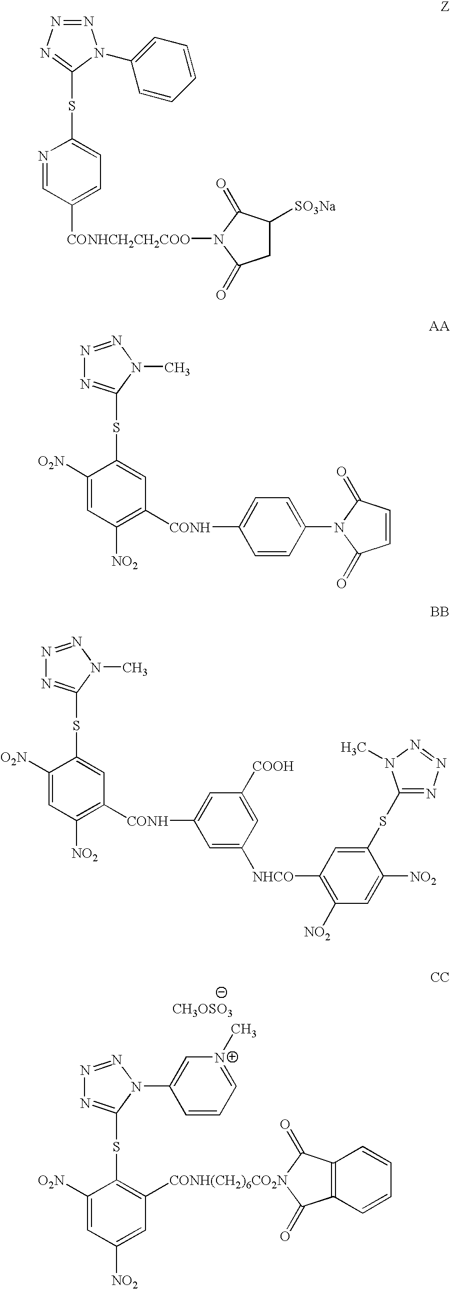

The conjugation agent can have a desirable balance between selectivity and reactivity, such that the conjugation agent is selective for a desired target, while maintaining a desirable reaction time. For example, the reactivity of the conjugation agent should be comparable in time to the reactivity of other known conjugating agents, for example, bis(vinylsulfonyl)methane (BVSM), and conjugating agents that incorporate N-alkylmaleimides, such as but not limited to N-ethylmaleimide (NEMI). The reaction rate of the conjugation agent can be increased by strong electron withdrawal from the aromatic ring. The total electron withdrawal, as estimated by summing the para-σ of each substituent, can be equal to or greater than 0.5, for example, greater than or equal to 0.7, or greater than or equal to 1.0.

The selectivity for thiol-containing groups at the primary reaction site can be greater than or equal to 100,000, for example, greater than or equal to 1,000,000, or greater than or equal to 10,000,000.

The conjugation agent, alone or in a conjugate, can be stable in neutral, aqueous solution for at least 7 days, for example, at least 14 days, at least 20 days, or at least 24 days.

The conjugation agent described herein exhibits specificity for thiol-containing groups at the primary reaction site. The conjugation agent can link targets selected from biological materials, chemical materials, or a combination thereof. The conjugation agent can have high reactivity with and good selectivity for thiol-containing groups at the primary reaction site, and can be stable in aqueous solutions. The conjugation agent can be reactive with amines at the secondary reaction site.

### EXAMPLES

### Example 1

This example illustrates the combination of advantages of a conjugation agent of the invention as compared to those known in the art. The example examines reactivity towards thiol-containing groups, chemoselectivity for thiol-containing groups relative to amines, and aqueous stability of the conjugation agent.

The measures of thiol reactivities, thiol chemoselectivities, and aqueous stabilities were determined by the rate constants for the corresponding processes, i.e., relative rate constants for reaction with thiol-containing groups (thiol reactivity), the ratio of thiol to amine rate constants (chemoselectivity for thiol-containing groups), and the rate constant for decomposition of the non-bonded conjugation agent in an aqueous environment (aqueous stability). Equations used in the interpretation of kinetic experiments may be found in J. H. Espenson, Chemical Kinetics and Reaction Mechanisms; McGraw-Hill: New York, 1981.

For the reactivity measurements, sodium 3-mercapto-1-propanesulfonate (MESNA) was used as the thiol-containing target, and lysine (Lys) was used as the amine target. The relevant reaction sequences are shown below, where LG is a leaving group, such as a halide, or alternatively a leaving group of the invention (Sₓ—Rₛ), such as an N-ethylmercaptotetrazolyl (EMT) group. All reactions were carried out at standard temperature and pressure.

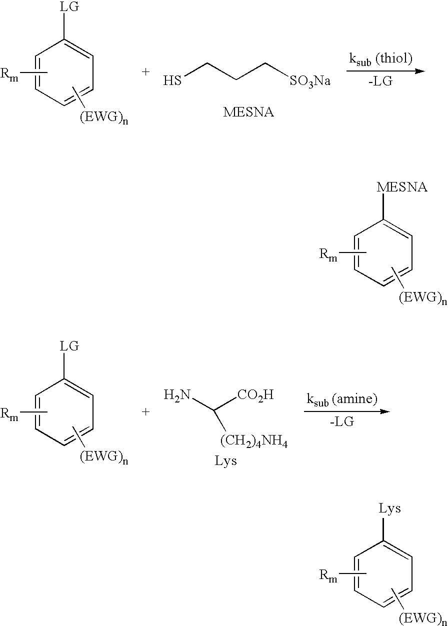

Table 1 shows a direct comparison between a range of substituted aromatics of the type known in the art and the invention. The general structure of the conjugation agents used in Example 1 is shown below in formula V, and details of the structure for each conjugation agent are provided in Table 1. All reactions were carried out in 80% phosphate buffer (pH 7)/20% acetonitrile for purposes of solubility. As shown in Table 1, the relative rate constant kᵣₑₗ is for a substitution reaction of the conjugation agent with thiol sodium 3-mercapto-1-propanesulfonate (MESNA). The selectivity is the ratio of kᵣₑₗ(MESNA)/kᵣₑₗ(Lys). The lifetime (τ) of a conjugation agent was measured as the time required for loss of about 66% of the starting amount in the solution of 80% phosphate buffer (pH 7)/20% acetonitrile. This demonstrates stability of the conjugation agent. The progress of the reactions was followed by high performance liquid chromatography (HPLC) for up to 1 week. For lifetimes longer than about one week, the values were estimated based on the observed extent of decomposition, where possible. Conjugates labeled “I” are inventive; those labeled “C” are comparative.

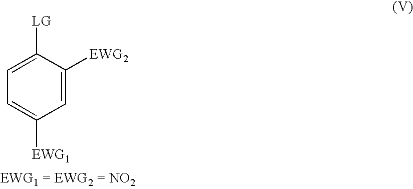

TABLE 1

| Conjugation | Leaving | | | |
|---|---|---|---|---|
| agent | group | kᵣₑₗ (MESNA) | Selectivity | Lifetime |
| I1 | EMT | 1 | >500,000 | nd |
| C1 | F | 2 | 722 | 17 days |
| C2 | Cl | 0.2 | 96,000 | nd |
| C3 | SO₃ | 0.001 | >500,000 | nd |

For I1 and C3, no reaction with lysine could be detected on the time scale of the experiment (1 week). Selectivity values were generated using the lowest detection limit of the equipment as the value of kᵣₑₗ(Lys). For I1, C2, and C3, hydrolysis was not detected (“nd”) on the time scale of the experiment (1 week).

The results set forth in Table 1 enable comparison between certain substituted aromatics of the type known in the art and the invention. The most reactive of the known comparison compounds is the fluoro-derivative C1, which showed twice the reactivity towards thiol-containing groups (kᵣₑₗ (MESNA)) than the representative compound of the invention I1, but also showed significantly higher reactivity towards amines, as shown by kᵣₑₗ (Lys) and the selectivity. C1 is also subject to hydrolysis. C1 therefore exhibits lower chemoselectivity than I1 and is unstable compared to I1.

The chloro-derivative C2 exhibited improved selectivity as compared to C1, but at the cost of reactivity towards thiol-containing groups. The rate constant for reaction with thiol-containing groups for C2 was an order of magnitude lower than that for C1. I1 was found to be five times more reactive towards thiol-containing groups than C2 and exhibited higher chemoselectivity.

The sulfonate C3 exhibited good chemoselectivity toward thiol-containing groups and hydrolytic stability, but had very low reactivity towards thiol-containing groups as compared to any of the other conjugation agents.

A further study with water soluble conjugation agents known in the art compared to those of the invention was conducted, and the results are shown in Table 2, including relative reactivity (kᵣₑₗ(MESNA)) and selectivity (kᵣₑₗ(thiol)/kᵣₑₗ(amine)) as determined for Table 1. The hydrolytic stability data was determined as the lifetime (r) as defined above, but in neutral (pH 7) aqueous phosphate buffer. Formulas for comparative conjugation agents C4-C10, and inventive conjugation agents I2-I6, are shown below.

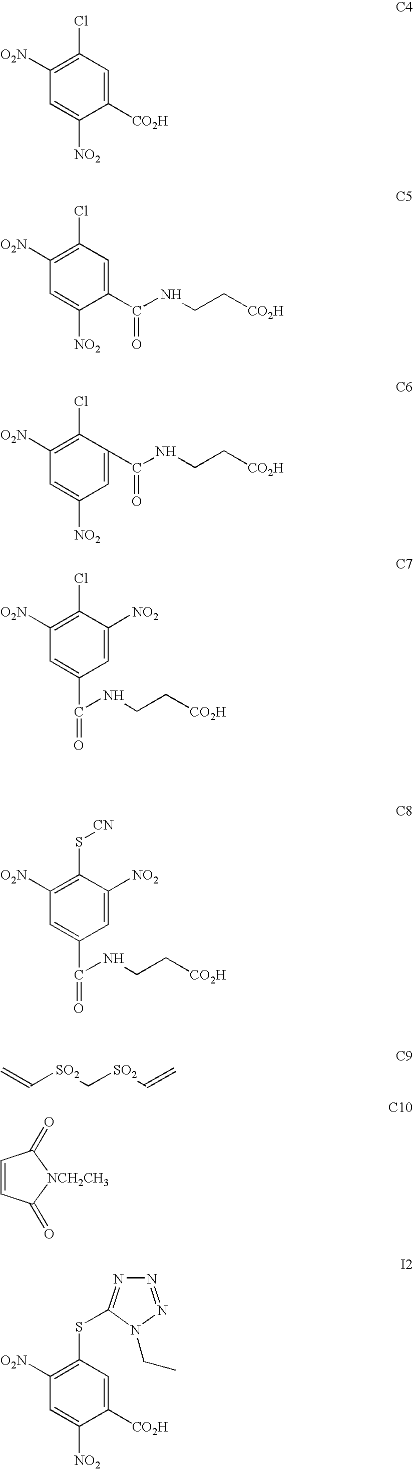

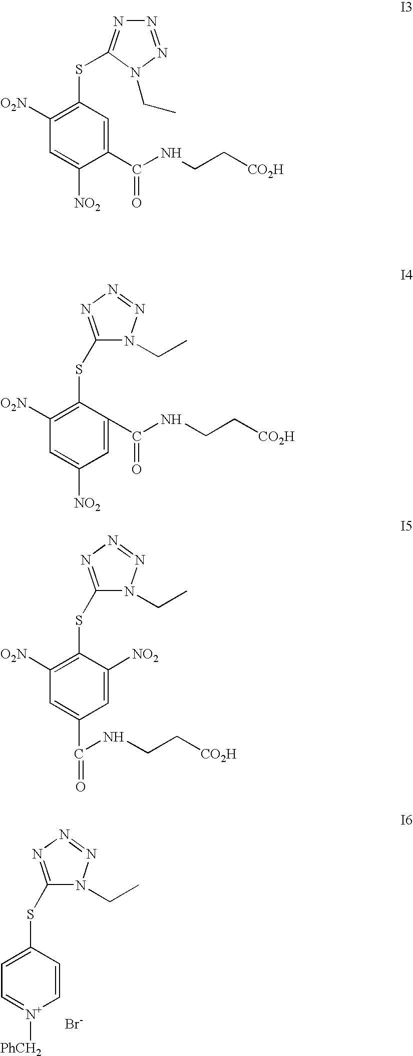

Comparison conjugation agents C4 through C7 are substituted aromatics containing functional group —CO₂H or —CONHCH₂CH₂CO₂H to improve water solubility. The analogous substituted aromatics that are representative of the primary reaction site of the invention are I2 through I5. C8 has a primary leaving group of SCN. C9 is bis(vinylsulfonyl)methane (BVSM), and C10 is N-ethylmaleimide (NEMI). N-ethylmaleimide is commonly used to react with thiol groups in biological applications and is available from Pierce Biotechnology Inc., Rockford, Ill. Because of their reactivity towards thiols, N-alkyl substituted maleimides like NEMI are commonly incorporated in conjugation agents for biosystems. An additional example of a commercially available conjugation agent containing N-alkylmaleimide, also available from Pierce Biotechnology Inc., is Sulfo-GMBS ((N-[γ-Maleimidobutyryloxy]sulfosuccinimide ester)). I6 is an example of the invention having an electron withdrawing ring.

TABLE 2

| | kᵣₑₗ (MESNA) | Selectivity | Lifetime | |
|---|---|---|---|---|
| C4 | 0.0003 | nd | nd | |
| C5 | 0.002 | nd | nd | |
| C6 | 0.004 | 4,200 | nd | |
| C7 | 0.006 | nd | 7 | days |
| C8^{f} | 5.2 | 112,000 | 3 | days |
| C9 | 0.7 | 538 | 18 | h |
| C10 | 24.3 | 867,000 | 1 | day |
| I2 | 0.008 | nd | nd | |
| I3 | 0.1 | nd | nd | |
| I4 | 0.4 | 369,000 | nd | |
| I5 | 1.0 | 178,000 | nd | |
| I6 | 1.9 | 10,000,000 | 24 | days |

^{f}HPLC analysis indicated multiple reaction products.

Within the subset of substituted aromatics, defined by I2 through I5 and C4 through C7, the observed reactivity trends shown in Table 2 serve to illustrate the influence of substituents and substitution pattern on reactivity. It is known that changing the nature of the substituent may affect reactivity. Thus, in each subset, i.e., among conjugation agents of the invention and among the comparison conjugation agents, substitution with amido solubilizing groups of the type —CONHCH₂CH₂CO₂H was found to provide improved reactivity towards thiol-containing groups relative to substitution with carboxylic acid solubilizing groups (—CO₂H). For example, kᵣₑₗ(thiol) for 13>kᵣₑₗ(thiol) for 12, and kᵣₑₗ(thiol) for C5>kᵣₑₗ(thiol) for C4. The effect of substitution pattern is illustrated by reactivity profiles of the isomers of the invention (I3, I4, and I5) and of the comparison conjugation agents (C5, C6, and C7). In both cases, the same substitution pattern (I5, C7) was found to provide the highest reactivity towards thiol-containing groups. However, the magnitudes of the substituent effects and of the substitution pattern effects were significantly greater in the case of the conjugation agents of the invention relative to the comparison conjugation agents. For example, kᵣₑₗ(thiol, I5)/kᵣₑₗ(thiol, 12) was about 125, whereas kᵣₑₗ(thiol, C7)/kᵣₑₗ(thiol, C2) was about 20.

Comparison of conjugation agents of the invention relative to the corresponding comparative conjugation agents, for example, comparing I2 to C4, I3 to C5, I4 to C6, and I5 to C7, demonstrates that the conjugation agent of the invention was significantly more reactive towards thiol-containing groups than the comparative conjugation agent. For example, I4 was found to be 100 times more reactive towards thiol-containing groups than C6. I4 was also significantly more selective for thiol-containing groups, about 88 times more selective, than C6. No difference was detected in their stabilities.

It was found the pyridinium reagent 16 provided higher selectivity for thiol-containing groups over amines (selectivity=10⁷) and enhanced reactivity as compared to the other conjugation agents of the invention.

C8 shows the effect of using a thiocyanate leaving group, as known in the art. This conjugation agent is found to exhibit good reactivity towards thiol-containing groups and selectivity, but it is significantly less stable than the conjugation agents of the invention. Analysis of the corresponding reaction mixture via high performance liquid chromatography (HPLC) indicated the formation of multiple reaction products, which is undesirable. The maleimide C10 is another example of a conjugation agent with high reactivity towards thiol-containing groups and good selectivity, but poor stability towards hydrolysis compared to conjugation agents of the invention.

The activated olefin bis-(vinylsulfonyl)methane C9 provides an example of a class of cross-linker. Whereas C9 has sufficient reactivity towards thiols, comparable to those of the invention, it exhibits significantly lower selectivity and lower stability under the same conditions.

The data shown in Tables 1 and 2 demonstrates the improved reactivity with and selectivity for thiol-containing groups, and improved stability with regard to hydrolysis, of conjugation agents of the invention as compared to those known in the art.

### Example 2

Inventive conjugation agent I7 is an example of a conjugation agent designed to sequentially link a substrate containing amine groups, such as a support coated with gelatin, to a target containing thiol groups. 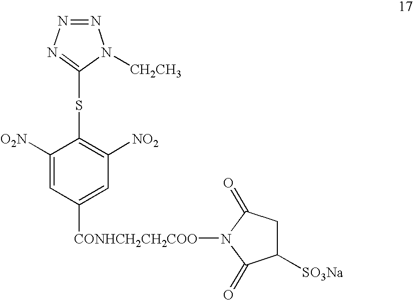
I7 was formed by conversion of the carboxylic acid functionality in I5 into a sulfosuccinate ester via standard procedures shown below in Example 3. This provided a functional group that was reactive towards amine groups. Near quantitative reaction of I7 with a corresponding lysine amide was conducted under transamination conditions (ambient temperature, pH 5.65, bimolecular rate constant for transamination, k_{bi}=3.3 M⁻¹min⁻¹). This reaction was carried out under mildly acidic conditions to minimize the known competing hydrolysis of the sulfosuccinate ester. The resultant conjugate was called I8.

I8 was reacted quantitatively with thiol-containing target MESNA (ambient temperature, pH 5.65). The amide conjugate I8 was found to be even more reactive (1.6 times) towards thiols than the closely related conjugation agent I5 based on relative bimolecular rate constants for reaction with MESNA under the same conditions. No hydrolysis or substitution of the EMT group in I8 by lysine was evident under the experimental conditions.

### Example 3

An exemplary reaction scheme for preparation of exemplary conjugation agents I5, I7, and C7 is presented below. Other conjugation agents can be prepared via these and other similar reactions transformations known in the art.

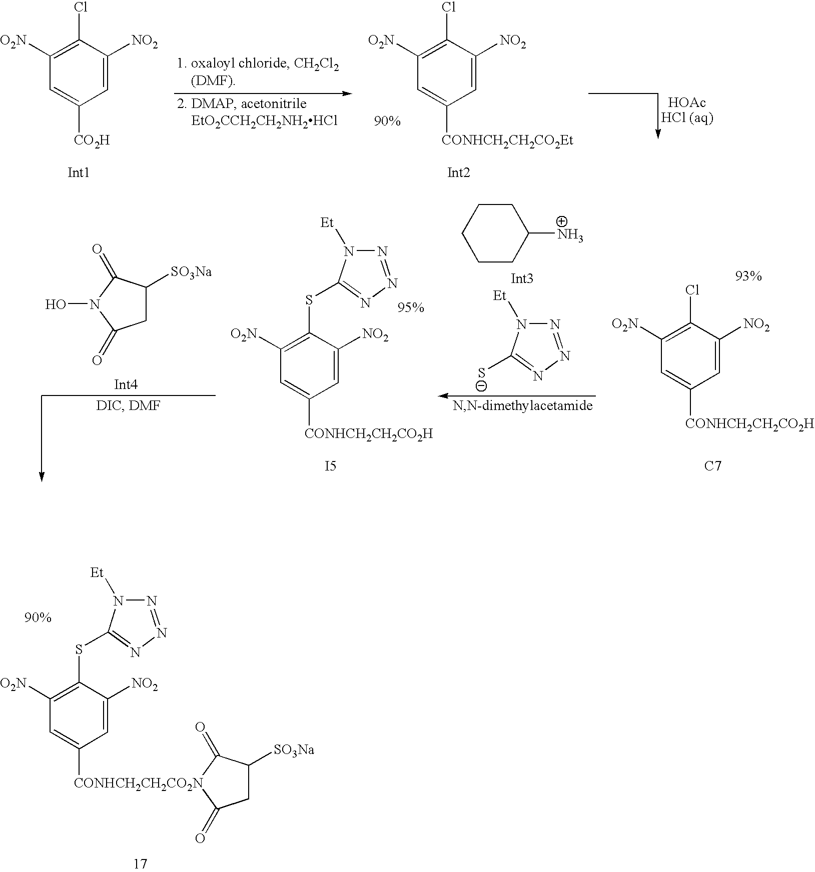

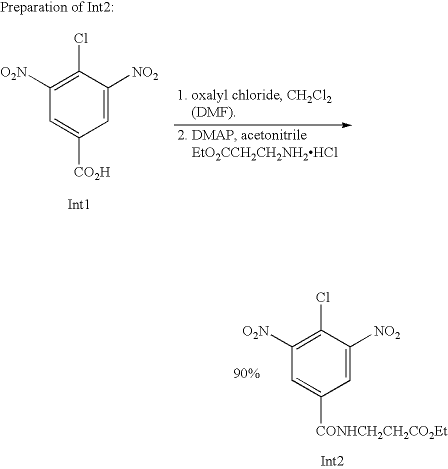

A slurry of 4-chloro-3,5-dinitrobenzoic acid, Int 1 (CAS 118-97-8; 24.6 g, 0.10 mol), in 200 mL of dichloromethane was treated with oxalyl chloride (10 mL) followed by addition of two drops N,N-dimethylformamide (DMF) as a catalyst, which induced vigorous gas evolution. As gas evolution slowed, an additional drop of catalyst was added. This procedure was repeated three times. After the final addition of catalyst, the mix was stirred at ambient temperature for thirty minutes. The mix was concentrated in vacuo. Heptanes were added and stripped off (4×100 mL of heptanes) to provide 4-chloro-3,5-dinitrobenzoyl chloride (crude product; 26.7 g, ca. 100%).

A solution of ethyl 3-aminopropionate hydrochloride (CAS 4244-84-2; 4.00 g, 26 mmol) and 4-dimethylaminopyridine (DMAP; 6.10 g, 50 mmol) in 100 mL of acetonitrile was chilled in an ice bath then treated with 4-chloro-3,5-dinitrobenzoyl chloride (6.62 g, 25 mmol) at once. The cold mixture was stirred for 30 minutes then poured into 500 mL of cold, dilute hydrochloric acid (10 wt %). The resulting slurry was filtered. The solid was washed with minimal deionized water and air dried to provide Int2 as a yellow solid (7.72 g, 90%). This material was chromatographically homogenous and displayed spectral characteristics consistent with its assigned structure.

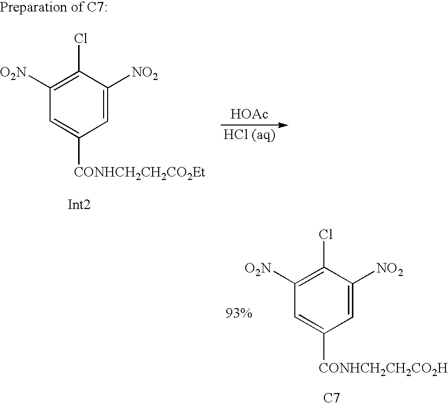

A slurry of Int2 (7.5 g, 21.7 mmol) in 80 mL of acetic acid was treated with 20 mL of concentrated hydrochloric acid. The resulting mixture was heated at 55-60° C. for 1.5 hours. The reaction was placed in about 0.5 L ice water and stirred. The resultant slurry was filtered and air-dried briefly. The damp solid was dissolved in ethyl acetate, dried, and concentrated in vacuo. The residue was triturated with I50 mL of refluxing isopropyl ether (IPE), cooled, and filtered. The resulting solid was washed with minimal IPE to provide C7 as a pale yellow solid (6.42 g, 93%). This material was chromatographically homogenous and displayed spectral characteristics consistent with its assigned structure.

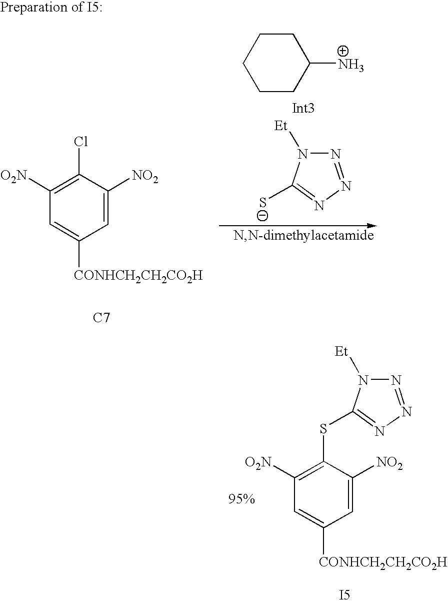

A solution of C7 (6.00 g, I8.9 mmol) in 75 mL of N,N-dimethylacetamide (DMAc) at ambient temperature was treated with Int3 (4.54 g, 1.98 mmol), which was readily prepared by reaction of 1-ethyl-5-mercapto-1,2,3,4-tetrazole (EMT; CAS I5217-53-5) with aminocyclohexane (CAS 108-91-8) in an inert solvent. The condensation reaction was stirred for 30 minutes then poured into 0.5 L water. The resulting slurry was filtered and the isolated solid washed with minimal water and air-dried to afford I5 as a pale yellow solid (7.35 g, 95%). This material was chromatographically homogenous and displayed spectral characteristics consistent with its assigned structure.

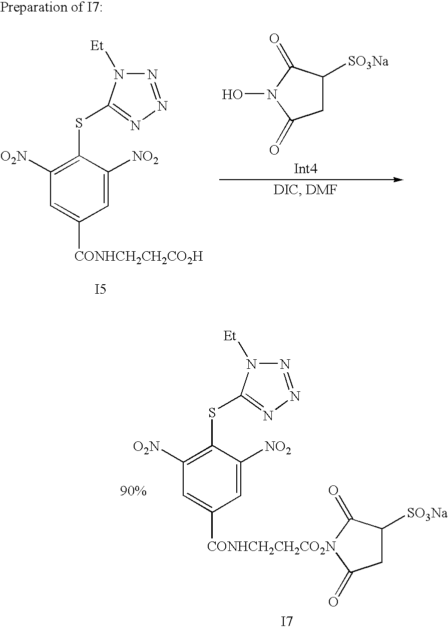

A mixture of I5 (6.16 g, I5.0 mmol), sodium N-hydroxysulfosuccinimide (Int4 (see below); 3.25 g, I5.0 mmol), and diisopropylcarbodiimide (DIC; 2.5 mL, 16.0 mmol) in 50 mL of DMF was stirred at ambient temperature for 20 hours, then filtered through diatomaceous earth. The solid materials were washed with minimal DMF. The combined filtrates were concentrated in vacuo employing a xylene azeotrope (3×100 mL) at 30° C. The residue was triturated with 50 mL of acetonitrile. The mix was filtered to remove any impurity, and then the filtrate was concentrated. The residue was triturated with 200 mL of ethyl acetate to provide a crude solid. The solid was heated to reflux in a further 200 mL of ethyl acetate, cooled to ambient temperature, and filtered to afford I7 as a yellow solid (3:1 ethyl acetate impurity; 8.28 g, 90%). This material was chromatographically homogenous and displayed spectral characteristics consistent with its assigned structure.

To prepare I7, Int 4 was required. The preparation of Int 4 was as follows. 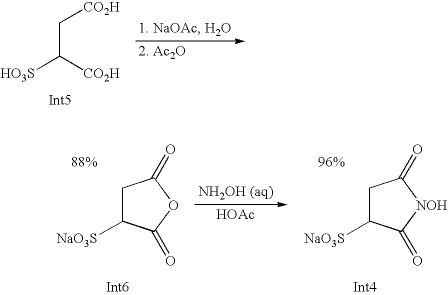 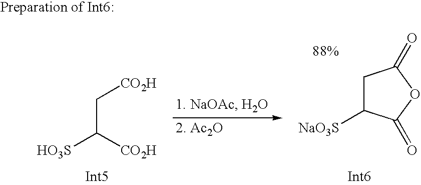
A solution of commercially available sulfosuccinic acid (Int5, 70% aqueous solution; 141.5 g, 0.50 mol) in a 100 mL of water was treated with sodium acetate (41.0 g, 0.50 mol). The mixture was stirred at ambient temperature until homogenous, about 10 minutes, then concentrated in vacuo. The viscous residue was azoetropically dried using acetonitrile (3×150 mL) distilled in vacuo to provide sodium sulfosuccinate as a colorless solid (109.1 g, 99.2%).

This solid was suspended in 300 mL of acetic anhydride and heated at 140° C. for two hours, then cooled to ambient temperature. The resulting slurry was filtered. The solids were washed with minimal acetic acid, then with IPE (3×100 mL) to yield sodium sulfosuccinic anhydride, Int6, as a colorless solid (97.1 g, 88%). This material was chromatographically homogenous and displayed spectral characteristics consistent with its assigned structure.

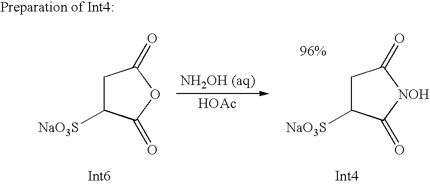

A slurry of Int6 (95.0 g, 0.470 mol) in 1250 mL of acetic acid was treated with commercial aqueous hydroxylamine solution (50% aqueous solution; 29 mL, 0.47 mol) then mechanically stirred at ambient temperature for 30 minutes. The resulting thick slurry was heated at 80-85° C. (external temperature) for I5 hours, and then cooled to room temperature. The slurry was filtered, washed with 0.5 L IPE in portions, then air-dried to give sodium N-hydroxysulfosuccinimide, Int4, (97.7 g, 96%). This material was chromatographically homogenous and displayed spectral characteristics consistent with its assigned structure.

The invention has been described in detail with particular reference to certain preferred embodiments thereof, but it will be understood that variations and modifications can be effected within the spirit and scope of the invention.

1. A conjugation agent of the formula: 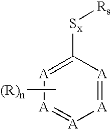
wherein Sₓ is —S—, —SO— or —SO₂—; Rₛ is a carbon-containing substituent that does not include a cyano group; each R is a substituent, with at least one R having a reaction site that is not a carboxylic acid; n is 0-5; each A is carbon or nitrogen, with the proviso that no more than three A can be nitrogen; and wherein Sₓ—Rₛ is a leaving group.

2. The conjugation agent according to claim 1, wherein Sₓ is —S—.

3. The conjugation agent according to claim 1, wherein Rₛ is a carbocyclic aromatic.

4. The conjugation agent according to claim 1, wherein Rₛ is a substituted or unsubstituted heterocycle.

5. The conjugation agent according to claim 1, wherein Rₛ is a five-membered substituted or unsubstituted heterocycle.

6. The conjugation agent according to claim 1, wherein Rₛ contains a nitrogen.

7. The conjugation agent according to claim 1, wherein Rₛ is a tetrazole, pyrazole, imidazole, or triazole.

8. The conjugation agent according to claim 1 that is homofunctional.

9. The conjugation agent according to claim 1 that is heterofunctional.

10. The conjugation agent of claim 1, having the formula: 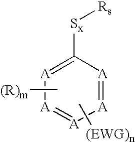
wherein Sₓ is —S—, —SO— or —SO₂—; Rₛ is a carbon-containing group that does not include a cyano group; each R is a substituent, with at least one R having a reaction site that is not a carboxylic acid; each EWG is a substituent that includes an electron withdrawing group; n is 0-5; m is 0-4, m+n is less than or equal to 5; each A is carbon or nitrogen, with the proviso that no more than three A can be nitrogen, and the proviso that when all A are carbon n is greater than or equal to 1; and wherein Sₓ—Rₛ is a leaving group.

11. The conjugation agent of claim 10, wherein Sₓ is —S—.

12. A conjugation agent according to claim 10, wherein m is 1.

13. A conjugation agent according to claim 10, wherein n is 2.

14. A conjugation agent according to claim 10, wherein each EWG is independently selected from a substituent containing a nitro group, a cyano group, a nitroso group, a sulfonyl group, a carbonyl group, or a sulfoxyl group.

15. A conjugation agent according to claim 10, wherein at least one EWG is a substituent containing a nitro group.

16. A conjugation agent of claim 1, having the formula 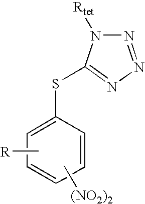
wherein Rₜₑₜ is a substituted or unsubstituted C1-10 alkyl, a substituted or unsubstituted C5-10 carbocycle, or a substituted or unsubstituted C5-10 aromatic.

17. A conjugation agent of claim 1, having the formula 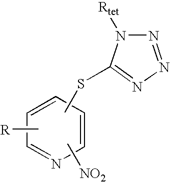
wherein Rₜₑₜ is a substituted or unsubstituted C1-10 alkyl, a substituted or unsubstituted C5-10 carbocycle, or a substituted or unsubstituted C5-10 aromatic.

18. A conjugation agent of claim 1, having the formula 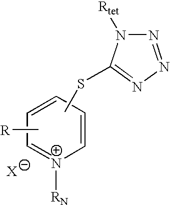
wherein Rₜₑₜ is a substituted or unsubstituted C1-10 alkyl, a substituted or unsubstituted C5-10 carbocycle, or a substituted or unsubstituted C5-10 aromatic, R_{N} is a substituted or unsubstituted C1-12 alkyl, and X⁻ is an anion.

19. A conjugation agent of claim 1, having the formula 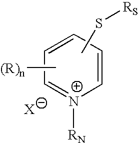
wherein R_{N} is a substituted or unsubstituted C1-12 alkyl.

20. A method of forming a conjugate comprising two or more targets and at least one conjugating agent, the method comprising obtaining the two or more targets, and bringing the targets into contact with the at least one conjugation agent of claim 1.

21. The method of claim 20, wherein the targets are the same.

22. The method of claim 20, wherein the targets are different.

23. The method of claim 20, wherein the at least one conjugation agent contacts one target at a time.

24. The method of claim 20, wherein more than one conjugation agent is present.

25. The method of claim 24, wherein the conjugation agents are different.

26. The method of claim 20, wherein the conjugate has the formula
X—(L)ₚ—Y
wherein each L is one conjugation agent, and each L can be the same or different from at least one other L; X and Y are each independently targets to be joined by L; and p is 1-4.
